(19) United States
(12) Plant Patent Application Publication
(10) Pub. No.: US 2003/0126653 P1
(43) Pub. Date: Jul. 3, 2003

(54) PISTACHIO ROOTSTOCK NAMED 'PN 15-4'

(76) Inventors: Henry P. Anderson III, Tulare, CA (US); Kenneth L. Puryear, Visalia, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/035,680

(22) Filed: Dec. 28, 2001

(51) Int. Cl. A01H 5/00

(52) U.S. Cl. PLT/152

(57) ABSTRACT

A new and distinct variety of pistachio tree, characterized by the deep, dark red color of young foliage, the rich dark green color of mature foliage, and the rounded upright growth habit in young trees.

### BACKGROUND OF THE INVENTION

The present invention relates to a new and distinct variety of pistachio tree, which is a hybrid formed from the crossing of Pistachio atlantica as seed parent with P. integerrima as pollen parent. The varietal denomination of this new pistachio is ‘PN 15-4’.

### SUMMARY OF THE INVENTION

The ‘PN 15-4’ variety is characterized by small, leathery leaves, with new glossy leaflets and older leaflets that are semi-glossy to dull. New leaflets are a deep, dark red and the older leaflets are rich, dark green. Centers of newer leaves are red and centers of older leaves are reddish green.

Growth of the variety is rounded and upright when young. The trunk is generally round, and mature growth is light brown in color, while new growth is reddish. Vegetative buds are small and reddish on new growth and larger reddish brown on older growth.

The variety is particularly useful as a rootstock upon which commercial nut bearing pistachio varieties may be budded or grafted.

Asexual reproduction by budding onto P. integerrima at Kern County, California shows that the foregoing and other distinguishing characteristics come true to form and are established and transmitted through succeeding propagation. Other known forms of asexual reproduction for pistachios may also be employed.

### BRIEF DESCRIPTION OF ILLUSTRATIONS

The accompanying photographic illustrations show typical specimens of vegetative growth of the new variety, in color as nearly true as it is reasonably possible to make in a color illustration of this character.

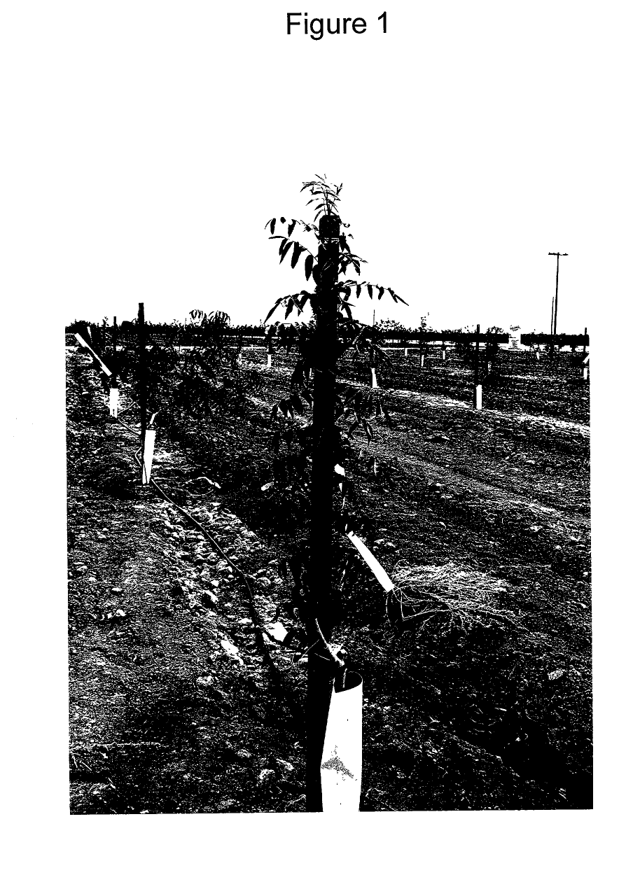

FIG. 1 shows a one year old pistachio tree of the new variety.

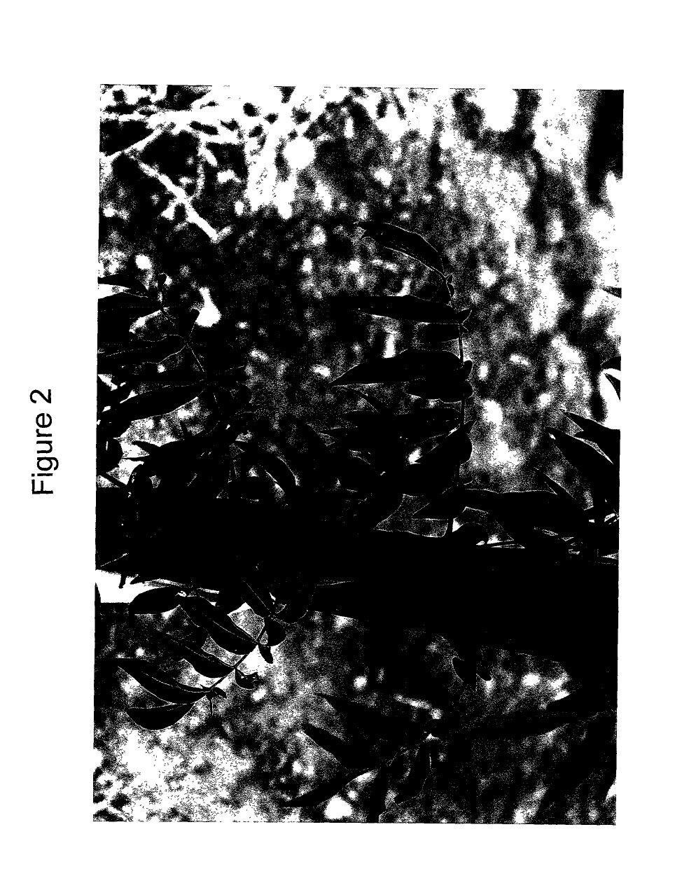

FIG. 2 shows a close-up of the trunk and compound leaves, typically with eleven leaflets.

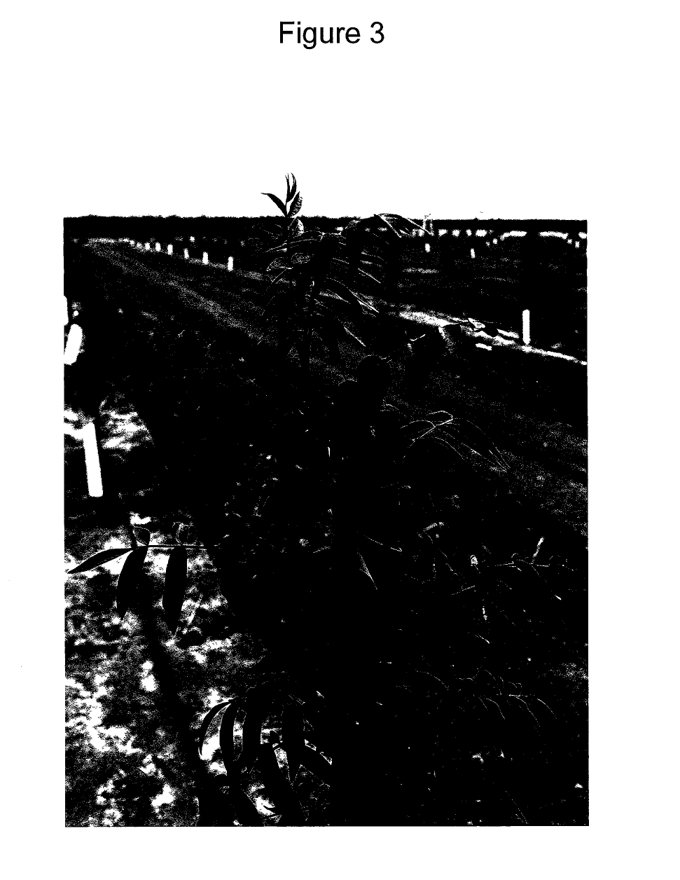

FIG. 3 shows a close-up of the new variety with both dark red new leaflets and dark green older leaflets.

### DESCRIPTION OF THE NEW VARIETY

Referring now specifically to the new and distinct variety of pistachio tree, the following is a detailed description of one year old plants with color description where indicated by reference to The Royal Horticultural Society Colour Chart, except where common terms of color definition are employed.

The new variety of pistachio tree as herein described may vary in slight detail due to climatic, soil and cultural conditions under which the variety may be grown, the present description being of the variety as grown at Kern County, California.

Foliage:

Leaves.—Compound of eleven (sometimes less), as indicated in FIG. 2.

Leaflets.—Small, leathery, with new leaflets glossy and older leaflets semi glossy to dull. Shape: medium length and narrow (lanceolate) apex acute, base acute but less so than apex. Color: new leaflets a deep dark red, near Greyed-red Group 178A, with older leaflets a rich dark green, near Greyed-green Group 189A, as shown in FIG. 3. Ribs: (Centers) on leaves are red, near Greyed-red Group 178B, in newer leaves to reddish green, near Greyed-red Group 181B, in mature leaves. In mature leaves, the center rib of leaflets and the leaflet veins are lighter green, near Greyed-green Group 189C, than the leaflets. In newer leaves, the center rib of the leaflet and the leaflet veins are lighter red, near Greyed-red Group 182C, than the leaflets.

Length of leaves (including petiole and leaflets).—May average about 8 inches and about 5.5 inches to end of midrib. Individual leaflets may average up to about 3 inches.

Growth:

Habit.—Rounded and upright when young.

Trunk.—Trunk is rounded. Light brown in color, near Greyed-orange Group 175A, on older growth and deep red, near Greyed-red Group 178A,in newer growth. Vegetative buds are small and reddish on new growth with larger reddish brown buds on older growth.

We claim:

1. A new and distinct variety of pistachio tree, identified as ‘PN 15-4,’ substantially as shown and described.
